(19) United States
(12) Patent Application Publication
(10) Pub. No.: US 2016/0252517 A1
(43) Pub. Date: Sep. 1, 2016

(54) BIOMOLECULAR INTERACTION DETECTION DEVICES AND METHODS

(71) Applicants: THE REGENTS OF THE UNIVERSITY OF CALIFORNIA, Oakland, CA (US); NATIONAL TAIWAN UNIVERSITY, Taipei (TW)

(72) Inventors: Yu-Hwa Lo, San Diego, CA (US); Tiantian Zhang, San Diego, CA (US); Jian Jang Huang, Taipei City (TW); Shou-Hao Wu, Taipei (TW)

(21) Appl. No.: 15/028,634

(22) PCT Filed: Oct. 10, 2014
(86) PCT No.: PCT/US14/60175

§ 371 Date: Apr. 11, 2016

Related U.S. Application Data
(60) Provisional application No. 61/890,178, filed on Oct. 11, 2013.

(51) Int. Cl. G01N 33/68; B01L 3/00; G01N 33/94; G01N 27/414

(52) U.S. Cl. CPC G01N 33/6845; G01N 27/4145; B01L 3/502715; B01L 3/502738; G01N 33/94; B01L 2300/0819; B01L 2300/0645; B01L 2300/0864; B01L 2300/0887

(57) ABSTRACT

Methods, systems, and devices are disclosed for detecting molecular interactions. In one aspect, a device includes a substrate formed of an electrically insulative material, the substrate structured to form (i) a molecular deposition chamber to receive one or more fluid samples including biomolecules, in which the biomolecules are capable of undergoing molecular interactions in the molecular deposition chamber that changes a molecular property of the molecular-interacted biomolecules, and (ii) a microfluidic channel to carry the biomolecules, which, based at least partly on the molecular interactions, the biomolecules travel through the microfluidic channel with different diffusivities; and an electronic sensor including an electrode configured along or at one end of the microfluidic channel and a transistor to detect the changed molecular property of the molecular-interacted biomolecules as a change in electrical signal, in which the electronic sensor is operable to produce an output signal corresponding to the detected electrical signal.

### CROSS-REFERENCE TO RELATED APPLICATIONS

This patent document claims benefit of priority of U.S. Provisional Patent Application No. 61/890,178, entitled “BIOMOLECULAR INTERACTION DETECTION DEVICES AND METHODS” and filed on Oct. 11, 2013. The entire content of the aforementioned patent application is incorporated by reference as part of the disclosure of this patent document.

### TECHNICAL FIELD

This patent document relates to molecular sensor technologies for sensing biological substances, chemical substances and other substances.

### BACKGROUND

Sensors based on electrochemical processes can be used to detect a chemical, substance, a biological substance (e.g., an organism) by using a transducing element to convert a detection event into a signal for processing and/or display. Biosensors can use biological materials as the biologically sensitive component, e.g., such as biomolecules including enzymes, antibodies, nucleic acids, etc., as well as living cells. For example, molecular biosensors can be configured to use specific chemical properties or molecular recognition mechanisms to identify target agents. Biosensors can use the transducer element to transform a signal resulting from the detection of an analyte by the biologically sensitive component into a different signal that can be addressed by optical, electronic or other means. For example, the transduction mechanisms can include physicochemical, electrochemical, optical, piezoelectric, as well as other transduction means.

### SUMMARY

Disclosed are methods, systems, and devices to detect and characterize molecular interactions including protein-ligand and protein-protein interactions.

In one aspect, a high-throughput molecular interaction detection device includes a substrate including an electrically insulative material and structured to form (i) an array of wells to receive corresponding fluid samples including candidate molecules, and (ii) a microfluidic channel positioned above openings of the wells, in which the microfluidic channel is shaped to carry a fluid including target biomolecules to the openings of the wells to create fluid interfaces between the fluid and the fluid samples; an electrode disposed on a surface of each well to detect a change in an electric signal based at least partly on molecular interactions between the target biomolecules and candidate molecules in a respective well; and a plurality of transistors electrically coupled to corresponding electrodes to generate an output signal based at least partly on the detected change in the electrical signal.

In one aspect, a device to detect molecular interactions includes a substrate including an electrically insulative material and structured to form a microfluidic channel to receive one or more fluid samples including biomolecules at a first region of the channel and to carry the fluid to a second region of the channel, in which the microfluidic channel is arranged on the substrate to enable a given biomolecule to undergo a molecular interaction with another given biomolecule that alters a molecular property of one or both the given biomolecule and the other given biomolecule to become a molecular-interacted biomolecule; an electrode disposed on a surface of the microfluidic channel in the second region to detect a change in an electrical signal based at least partly on molecular interactions of the biomolecules; and a transistor electrically coupled to the electrode to generate an output signal based at least partly on the detected change in the electrical signal.

In one aspect, a device to detect molecular interactions includes a substrate formed of an electrically insulative material, the substrate structured to form (i) a molecular deposition chamber to receive one or more fluid samples including biomolecules, in which the biomolecules are capable of undergoing molecular interactions in the molecular deposition chamber that changes a molecular property of the molecular-interacted biomolecules, and (ii) a microfluidic channel to carry the biomolecules, which, based at least partly on the molecular interactions, the biomolecules travel through the microfluidic channel with different diffusivities; and an electronic sensor including an electrode configured along or at one end of the microfluidic channel and a transistor to detect the changed molecular property of the molecular-interacted biomolecules as a change in electrical signal, in which the electronic sensor is operable to produce an output signal corresponding to the detected electrical signal.

In one aspect, a device to detect molecular interactions includes a molecular reaction chamber to receive one or more fluid samples including biomolecules, in which the biomolecules undergo molecular interactions in the chamber that changes a molecular property of the molecular-interacted biomolecules, a microfluidic flow module including a microfluidic channel to carry the biomolecules, where, based on the molecular interactions, the biomolecules travel through the microfluidic channel with different diffusivities, and an electronic sensing module to receive the biomolecules from the microfluidic flow chamber and detect the changed molecular property of the molecular-interacted biomolecules as an electrical signal change, in which the electronic sensing module produces an output signal corresponding to the detected.

In one aspect, a method to detect molecular interactions includes receiving a fluid sample including biomolecules in a microfluidic channel at a first region of the microfluidic channel to flow the fluid sample carrying the biomolecules through the microfluidic channel to a second region of the channel; detecting a change in an electrical signal at an electrode disposed on a surface of the microfluidic channel in the second region, in which the detected change in the electrical signal is based at least partly on molecular interactions among the biomolecules causing an induced surface charge on the electrode; and processing the detected change in the electrical signal to determine an occurrence of the molecular interactions among the biomolecules.

In one aspect, a method for high-throughput detection of molecular interactions includes receiving a plurality of fluid samples including candidate molecules in an array of wells formed on a substrate; receiving a fluid including target biomolecules in a microfluidic channel formed on the substrate in fluidic connection with the array of wells, in which the fluid carrying the target biomolecules from the microfluidic channel to openings of the wells create fluid interfaces between the fluid and the fluid samples; detecting a change in an electrical signal from an electrode disposed on a surface of a corresponding well, in which the detected change in the electrical signal is based at least partly on molecular interactions between the target biomolecules and candidate molecules causing an induced surface charge on the corresponding electrode; and processing the detected change in the electrical signal from each electrodes associated to the corresponding wells to determine an occurrence of the molecular interactions between the target biomolecules and the respective candidate molecules.

The subject matter described in this patent document can be implemented in specific ways that provide one or more of the following features. For example, the disclosed technology includes a device architecture and a methodology to enable investigation of protein-ligand and protein-protein interactions as well as fundamental protein properties in conditions close to the physiological environments. In implementations, for example, the disclosed techniques require no labeling of the molecules, and impose no constraints on the motions of the molecules under study. The disclosed techniques can be implemented to produce both qualitative (e.g., whether ligand-protein binding occurs or not) and quantitative information (e.g., the reaction constants), and is applicable to a large variety of proteins and ligands of different molecular weight, charge, hydrophobicity, and 3D configurations. The disclosed technology can be implemented in a variety of applications including high-throughput drug screening and research in biological sciences, among others.

### BRIEF DESCRIPTION OF THE DRAWINGS

FIG. 1A shows a block diagram of an exemplary biomolecular interaction detection device of the disclosed technology.

FIGS. 1B and 1C show block diagrams depicting exemplary embodiments of the biomolecular interaction detection device shown in FIG. 1A.

FIG. 2 shows a diagram illustrating detection of molecular interactions using an exemplary sensor module of an exemplary device of the disclosed technology.

FIG. 3A shows a diagram of an exemplary molecular binding implementation using the disclosed technology for ligand (e.g., smaller molecule) and protein (larger molecule) binding.

FIG. 3B shows a diagram of an exemplary molecular interaction implementation using the disclosed technology for protein-protein interactions.

FIG. 4A shows a schematic diagram of an exemplary embodiment of a biomolecular interaction detection device.

FIG. 4B shows a cross sectional diagram of a microfluidic channel of an exemplary device depicting the fluid distribution in the channel above a detecting electrode.

FIGS. 5A-5D show data plots of exemplary data measured depicting the detection and analysis of avidin-biotin interactions.

FIGS. 6A-6C show data plots of exemplary data measured depicting the detection and analysis of NADH-MDH interactions.

FIG. 7A shows a block diagram of an exemplary high-throughput biomolecular interaction detection device.

FIG. 7B shows a schematic diagram of an exemplary method to prepare and implement an exemplary high-throughput biomolecular interaction detection device.

FIG. 8 shows a schematic diagram of an exemplary biomolecular interaction detection device including TFTs in the sensor module.

FIG. 9 shows data plots depicting thin film transistor (TFT) signals for protein detection using exemplary devices with different microfluidic channel lengths.

FIG. 10 shows I-V data plots depicting the drain current variation by molecules in the fluid.

### DETAILED DESCRIPTION

For basic biological science and drug discovery, an important task and challenge is to understand protein-ligand binding and protein folding without external interference, e.g., since proteins have extraordinary flexibilities and their proper functions in living systems depend on correct folding and configurations. Additionally, protein-protein binding to form protein complexes and protein binding with small molecules provide important clues for drug discovery. Small molecules that bond with proteins may produce therapeutic effects by changing or correcting the protein behaviors to activate or inactivate them. For example, among the library of millions of small molecules, promising drug candidates are selected by protein-ligand binding tests. For example, experiments are typically conducted after the first screening by computer simulations. Because of the difficulties in modeling protein behaviors, however, the software screening often yields a very large number of drug molecule candidates for further tests. Due to the extremely high and rapidly increasing cost for drug testing in the late stage of drug development, pharmaceutical companies seek accurate, reliable, and high throughput devices and methods for screening of drug candidates in their drug discovery work flow.

For fundamental biological sciences, uncovering the protein-protein and protein-ligand interactions is an important step to understand the biological functions in normal and diseased states. All biological functions including gene expression, cell growth, metabolism, enzymatic reactions, and various diseases such as cardiovascular diseases, neural diseases, immune diseases, and cancer, involve protein interactions with small molecules, other proteins, ions, nucleic acids, and other biomolecules. Understanding these functions is central to the advance of biological science. However, in spite of the undisputable significance of the problem, there has been no approach available that can detect protein interactions without disturbing the molecules under test.

Some current methods involve fluorescent labeling, including the fluorescence resonance energy transfer (FRET) technique. Although fluorescent labeling enables visualization of the protein molecules with high signal-to-noise ratio and excellent spatial resolution when used in fluorescence microscopy, introduction of the fluorescent molecules may alter the protein properties, restrict protein folding, and affect its binding affinity or binding sites. Similar problems also exist in other labeling techniques such as labeling with Raman probes, quantum dots, magnetic beads, nanoparticles, etc.

Among the label-free techniques, surface plasmonics resonance (SPR) is one exemplary technique for protein-ligand detection. Although no labels are attached to the protein molecules for SPR, the ligand molecules have to be immobilized to a solid surface. To achieve high sensitivity and specificity, the protein-ligand binding sites have to be very close to the gold surface of the SPR setup, e.g., typically within 10 nm. This imposes strict constraints on the motions of proteins and their interactions with ligands. In biological systems, often times both the proteins and the ligands are free to move in space and enjoy the high degrees of freedom to find the binding sites to form the desired configurations. Some of the degrees of freedom are taken away in the SPR setup. Because of the lack of an interruption-free technique to study protein interactions, current methods may yield incorrect results, e.g., either suggesting ineffective drug candidates or missing promising ones.

Devices, systems, and methods are disclosed for detecting and characterizing protein-ligand and protein-protein interactions and fundamental protein properties in conditions close to the physiological environments.

The disclosed molecular interaction detection technology integrates a field-effect transistor sensing device with a microfluidic device to achieve label-free, constraint-free detection of protein properties. Systems and devices of the present technology can be scaled to enable use in applications for studying protein behaviors in a massive parallel manner, e.g., suitable for drug screening and, more generally, biological sciences.

In one aspect, a biomolecular interaction detection device of the disclosed technology can be structured to include a sensing electronic module with a property (e.g., current, voltage, threshold voltage, etc.) that changes when part of the device is in contact with the target molecules, and a microfluidic module in which the suspended molecules (e.g., proteins and ligands) react within the microfluidic chamber and diffuse through the channels. For example, different molecules and molecular complexes may have different diffusion speed inside the microfluidic channels, thus reaching the sensing electronic unit at different times. The arrival of each type of molecule at the sensing module gives rise to a change of its current or other properties. By measuring the arrival times of different molecules or molecular complexes and the amount of current changes caused by them, information about the target molecules can be obtained. In some implementations, the disclosed devices can be used for protein-ligand binding, protein-protein interaction, and protein folding and reconfiguration detection, as well as detection of other protein characteristics, e.g., such as denaturing, charge, and diffusivity.

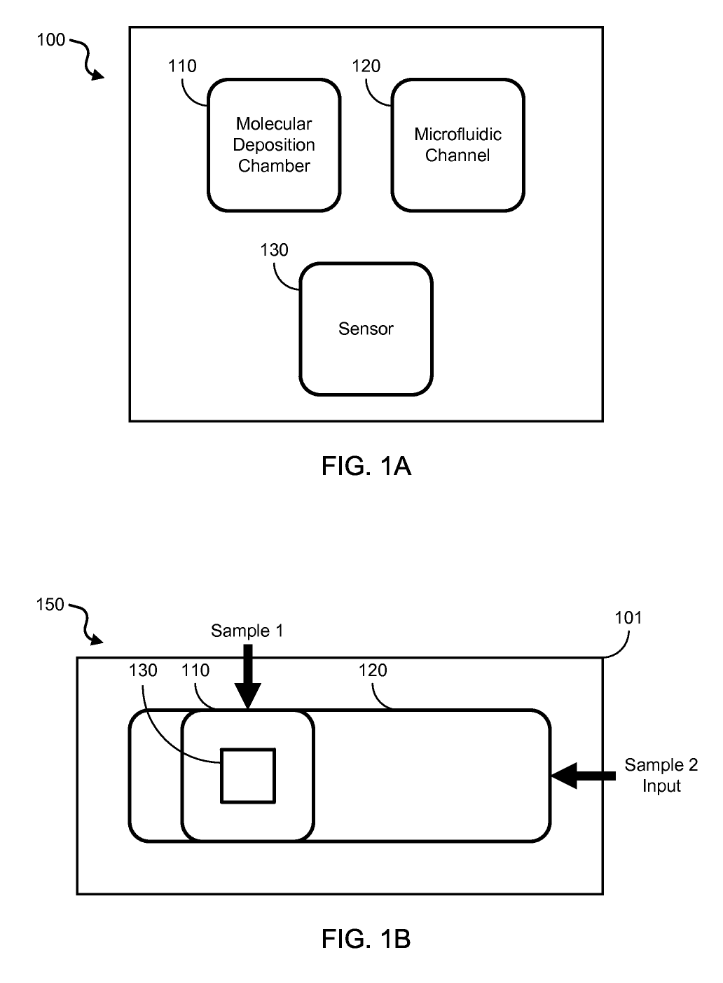

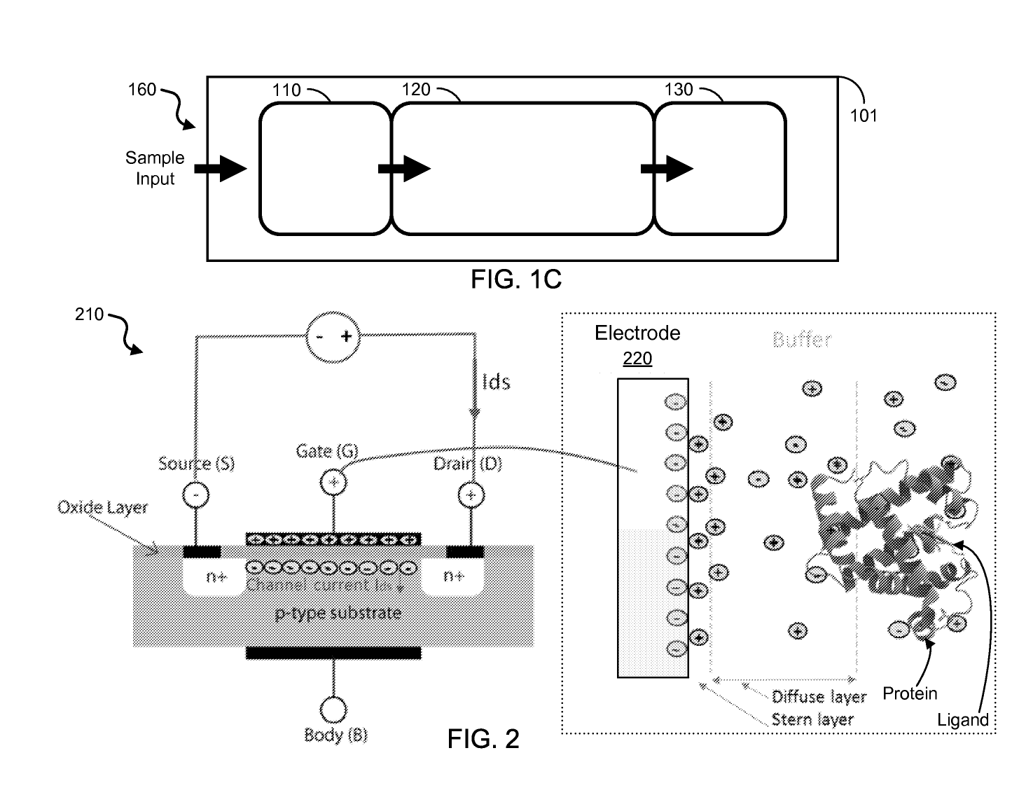

FIG. 1A shows a block diagram of an exemplary biomolecular interaction detection device 100. The device 100 includes a molecular deposition chamber 110 to receive one or more samples containing molecules in a fluid, e.g., such as ligands and/or proteins for detecting and characterizing protein-ligand and/or protein-protein interactions. The device 100 includes a microfluidic channel 120 to allow the molecules of the inputted sample to pass through according to their own molecular properties and kinetics to a sensor module 130 of the device 100. Due to the different kinetic properties among biomolecules, for example, different biomolecules are detected by the sensing module 130 of the device 100 at different times to produce signals corresponding to different types of molecules. These signals detected at the sensor 130 provide information about specific molecular binding, interactions, and morphological properties.

The sensor module 130 can include an electrode configured in the microfluidic channel 120 and/or molecular deposition chamber 110 and in electrical communication with an electronic circuit to achieve label-free, constraint-free detection of molecular properties, e.g., such as proteins. In some embodiments, for example, the electronic circuit can include a transistor coupled to an electrical meter, e.g., a source meter. The transistor may be a field-effect transistor (FET) with its gate electrically coupled to the electrode area exposed to the fluid in the microfluidic channel 120 and/or molecular deposition chamber 110. In some implementations, for example, this exposed electrode electrically coupled to the gate of the FET may include a surface functionalized or patterned metal, e.g., such as gold. In some implementations of the device 100, for example, the sensor module 130 includes one or more electrodes configured in the microfluidic channel 120 and/or molecular deposition chamber 110 connected to contact pads via electrical interconnects or vias, in which the contact pads are capable of electrically connecting to an external electronic circuit to determine the signals detected by the electrodes. Similarly, for example, in some implementations of the device 100, the sensing module 130 includes the FETs in electrical communication with the electrodes configured in the microfluidic channel 120 and/or molecular deposition chamber 110 on a single substrate, which is capable of electrically connecting to an external electronic circuit by contact pads (connected to the outputs of the FETs) to determine the signals detected by the electrodes.

In one example of operation of the device 100, when the charged molecules are nearby the detecting electrode of the sensor 130, through the gate of the FET the carrier density in the FET channel is altered, yielding an increase or decrease in the drain current. The amount of the current change can be converted into the change in the effective surface charge on the exposed gate following the relation:

$$Δ Q = \frac{{c}_{gs}}{{g}_{m}} Δ I \qquad ( 1 )$$

where ΔQ is the amount of charge induced by the molecules, ΔI is the measured current change, and C_{gs} and gₘ are the gate-to-source capacitance and transconductance of the FET under given bias. It is noted that although the transistor senses the presence of the molecules, the signal itself cannot distinguish molecules. For the sensor 100 to distinguish molecules and detect interactions, it relies on the integration of the sensor module 130 with the microfluidic module 120 to provide the needed additional information. In the example using the FET, the sensing gate area of the FET is connected with a microfluidic channel, e.g., via an electrode positioned in the channel. In some implementations, for example, the proteins and ligands of given concentrations can be premixed (e.g., in the molecular deposition chamber 110 positioned at one end of the channel) before introducing such premixed samples to the microfluidic channel 120 for detection by the sensor 130; whereas in other implementations, for example, the proteins and ligands of given concentrations are deposited at different regions of the microfluidic channel 120 and come into contact at a predetermined location of the microfluidic channel 120 to be detected by the sensor 130. In some implementations, for example, the device 100 can include a microvalve between the reaction chamber and the microfluidic channel. The valve can be closed for a certain time period and then opened to allow the molecules diffuse into the microfluidic channel.

FIG. 1B shows a block diagram of an exemplary embodiment of the biomolecular interaction detection device 100, shown as device 150. The device 150 includes a substrate 101 formed of an electrically insulative material and structured to form a well in the substrate to provide the molecular deposition chamber 110 for containing a first fluid sample including molecules for investigation with a second sample, e.g., such as ligands and/or proteins for detecting and characterizing protein-ligand and/or protein-protein interactions. For example, the substrate 101 can be formed of glass, polydimethylsiloxane (PDMS), or other electrically insulative material. The device 150 is structured to include the microfluidic channel 120 formed at one end of the substrate and extending over the molecular deposition chamber or well 110 to flow a second fluid sample and allow the second fluid sample to pass over the molecular deposition chambers 110. The second fluid sample includes target molecules for detecting and analyzing their interactions with the molecules in the first fluid sample contained in the molecular deposition chamber or well 110. In some embodiments, for example, the device 150 can include multiple molecular deposition chambers or wells 110 in the microfluidic channel 120, e.g., which can be arranged in a linear array perpendicular to the direction of the channel, or in other linear or nonlinear arrangements in the channel. The device 150 includes at least one sensor 130 configured in the in the molecular deposition chamber or well 110, e.g., such as at the bottom of the well, to detect signals corresponding to the molecular properties and kinetics of the molecules, e.g., capable of indicating interactions between the molecules of the first and second samples. For example, the device 150 can determine molecular interactions including binding and or conformational changes of the molecular entities. In some embodiments, for example, the device 150 can include multiple microfluidic channels 120 to flow multiple fluid samples over one or more molecular deposition chambers 110 for high-throughput applications to investigate multiple types or combinations of molecular interactions simultaneously.

FIG. 1C shows a block diagram of an exemplary embodiment of the biomolecular interaction detection device 100, shown as device 160. The device 160 includes the molecular deposition chamber 110 to receive one or more samples containing molecules in a fluid, e.g., such as ligands and/or protein for detecting and characterizing protein-ligand and/or protein-protein interactions. The device 160 includes the microfluidic channel 120 formed on the substrate proximate the molecular deposition chamber 110 to allow the molecules of the inputted sample to pass through the channel according to their own molecular properties and kinetics to the sensor module 130 of the device 160. In some implementations of the device 160, for example, the molecular deposition chamber 110 can facilitate reactions among the molecular entities in the inputted sample, e.g., such as binding and or conformational changes of the molecular entities. After the molecules are deposited, and in some implementations allowed to react or otherwise interact, the molecules are introduced to the microfluidic channel module 120 of the device 160. For example, due to different diffusivities and kinetics among molecules, the different molecules arrive at the sensing module 130 of the device 160 at different times to produce signals corresponding to different types of molecules. These signals detected at the sensor 130 provide information about specific molecular binding, interactions, and morphological properties.

In some implementations of the device 100, the sensor 130 can include a transistor amplifier, e.g., such as a metal-oxide-semiconductor field effect transistor (MOSFET), connected to the sensing electrode that is in contact with the aqueous solution to effectively detect the binding and binding kinetics (e.g., reaction rate) of the molecular entities, e.g., protein and/or ligand. For example, as ions in the buffer solution of the fluid sample move due to concentration gradients, the motions of these charge particles induce charge on the surface of the electrode of the sensor module 130 in the microfluidic channel 120. The ionic charge distribution within the Debye length induces a change in the surface charge density on the metal electrode according to the following relation:

σₛ=∈∈ₒEₛ (2)

where ∈ is the dielectric constant of the buffer solution, Eₛ is the electric field at the interface of the (Au) electrode, and σₛ is the charge density (C/cm²) on the surface of the electrode.

The change in the surface charge density on the metal electrode can be determined by the exemplary FET of the sensor module 130 of the device 100. FIG. 2 shows a diagram illustrating detection of molecular interactions using an exemplary sensor 130 of the device 100. As shown in the diagram, a MOSFET 210 is electrically coupled to an electrode 220 positioned in the microfluidic device 120 and/or the molecular deposition chamber 110. The diagram depicts a protein molecule interacting with a ligand molecule. The protein and the ligand each include their own charge characteristics affecting the surface charge density on the surface of the electrode 220. For example, assuming the MOSFET has its transconductance gₘ and gate capacitance C_{g} and there is no gate leakage current, the change in the drain current of the transistor can be represented as:

$$Δ {I}_{ds} ( t ) = \frac{- {Ag}_{m} {σ}_{s} ( t )}{{C}_{g}} \qquad ( 3 )$$

where A is the area of the electrode in contact with the buffer solution. Provided the gate leakage cannot be neglected over the time period of concern (e.g., 0.1 second to a few seconds), then the measured current change is modified as:

$$Δ {I}_{ds} ( t ) = - \frac{{Ag}_{m}}{{C}_{g}} {e}^{- t / {RC}_{g}} {{\int }_{0}}^{t} {e}^{τ / {RC}_{g}} {J}_{ion} ( τ ) d τ \qquad ( 4 )$$

where Jᵢₒₙ(t) is the ionic current density towards the surface of the electrode, and R is the gate leakage resistance. In the extreme case that the gate leakage current becomes dominant, Eq. 4 can be reduced to:

ΔI_{ds}(t)≅−AgₘRJᵢₒₙ(t) (5)

For example, Eq. (5) demonstrates a good approximation if the gate leakage resistance is less than 10¹² Ohm or the ion current response is in the order of second. The measured drain current change in the MOSFET transistor can give direct information about the charge distribution or ion flow in the solution, and the ion flow is determined by the diffusivity of protein and ligand molecules. In almost all situations, the ions in the buffer solution (e.g., such as Na+, K+, Cl−, etc.) have much greater diffusivity than proteins and ligands. Therefore, the diffusion process is mainly limited by the proteins and ligands as they are the rate limiting, charged particles in the system.

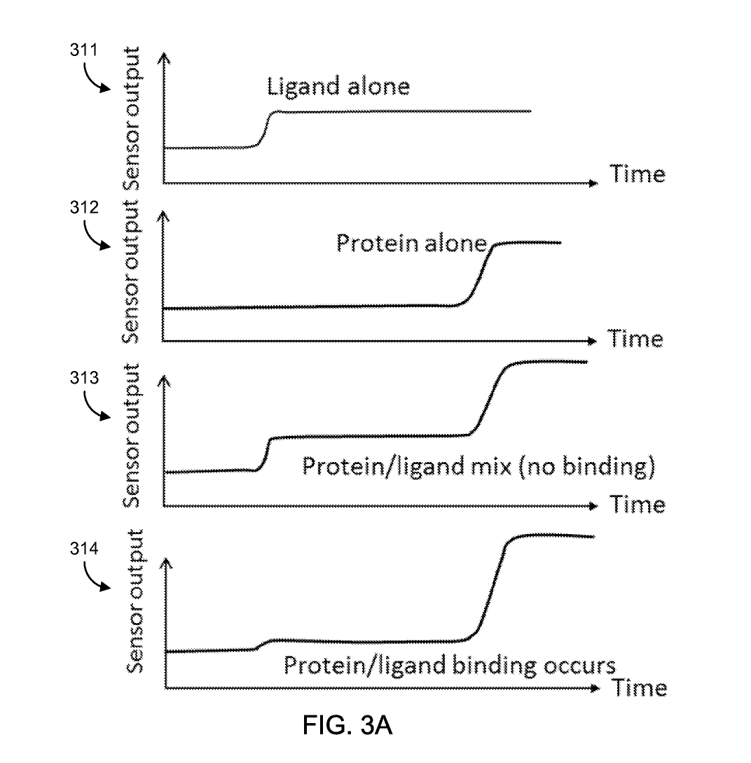

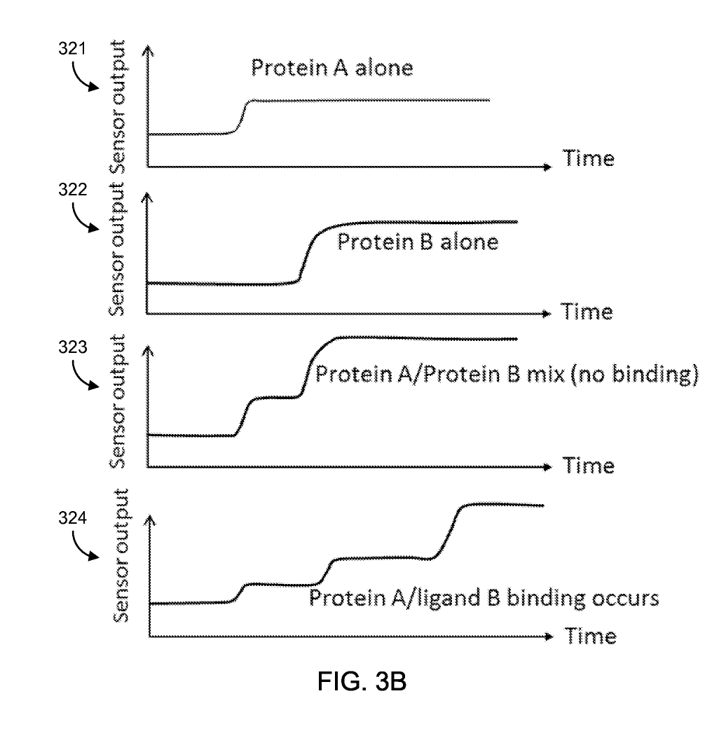

FIG. 3A shows a diagram of an exemplary molecular binding implementation using an exemplary device 100 of the disclosed technology to detect the interaction of ligand (e.g., smaller molecule) and protein (larger molecule) binding. FIG. 3A includes diagram 311 and 312 that show signals from the ligand alone and the protein alone, respectively. FIG. 3A includes a diagram 313 that shows the signal from a protein/ligand mix when binding does not occur. FIG. 3A includes a diagram 314 shows the signal from protein/ligand mixture when binding occurs.

In the exemplary implementation of FIG. 3A, three exemplary conditions were set up. For example, in the first two conditions, a given amount of ligand molecule (e.g., 10 μM) and protein molecule (e.g., 10 μM) were introduced separately; and in the third condition both protein and ligand were introduced together to allow protein-ligand interactions. After a certain period of time following the sample introduction to the exemplary molecular deposition chamber 110, e.g., such as in the device 160, an exemplary micro valve was opened and the molecules diffused through the microfluidic channel 120 to reach the sensing module 130 (e.g., electrode electrically coupled to a field-effect transistor). For example, assuming the ligand molecules are much smaller than the protein molecules (e.g., 1 kD for ligand and 50 kD for protein), one can expect to observe the characteristics shown in diagrams 311 and 312 in the first two conditions. The change of the FET current in the ligand-only test occurred earlier than the protein-only test because, for example, being a smaller molecule, a ligand has a greater diffusion coefficient which is related to the travel time according to the relation:

$$τ = \frac{{L}^{2}}{2 D} ,$$

where τ is the travel time of the molecule, D is the diffusivity, and L is the effective channel length.

For the third condition with a mixture of protein and ligand molecules, one may obtain different results depending on the binding affinity between the ligand and the protein. Provided the ligand and the protein do not bind together, the transistor signal will appear to be the superposition of the result from the ligand alone and the protein alone tests, as shown in diagram 313. On the other hand, if the ligand binds with the protein, the signal at the arrival time of the ligand will be diminished or disappear depending on the binding efficiency and the population ratio of ligand and protein, as shown in diagram 314. If the ligand-protein complex does not change the protein configuration significantly, the ligand-protein complex is expected to have a similar arrival time to the protein molecule by itself since the protein molecule is much greater than the ligand. However, if the binding does cause significant changes in protein configuration or folding, one may detect a different arrival time for the protein-ligand complex than the protein itself. Using the disclosed technology, based on the change in the ligand signal, one can obtain clear information whether the protein and the ligand form protein-ligand complex.

The disclosed systems, devices, and methods can be implemented in a myriad of applications for biological sciences and biotechnologies, and some of the exemplary applications are described next as examples.

Measuring Protein-Ligand Reaction Constant

The disclosed devices and methods can be implemented to obtain the reaction coefficient of protein-ligand binding. Although it may be difficult to measure the change of FET current between protein and protein-ligand complex since their magnitudes may be very close, the magnitude change in the ligand signal can be easy to detect. In the following example, described is an exemplary procedure to use the measured current change of the ligand to obtain the protein ligand reaction coefficient.

The protein ligand reaction can be represented as

P+L [symbol] PL (6)

For example, assume that initial concentrations of protein and ligand are [P] and [L] before reaction. After reaction and the equilibrium is reached, the following equation holds:

$${K}_{eq} = \frac{Δ}{( [ P ] - Δ ) ( [ L ] - Δ )} = \frac{1}{[ L ]} \frac{η}{\{ \frac{[ P ]}{[ L ]} - η \} \{ 1 - η \}} \\ η ≡ \frac{Δ}{[ L ]} \qquad ( 7 )$$

where K_{eq} is the equilibrium coefficient and Δ is the concentration of protein-ligand complex assuming a first order reaction (e.g., only one ligand molecule may bind with a protein molecule and the probability of multiple ligand molecules bond to a single protein is negligible). By measuring the change of the FET current that is proportional to the change of ligand concentration, obtained is

$$η = \frac{Δ}{[ L ]}$$

in Eq. (7). For example, with 10 μM of ligand molecules, obtained is a ligand-induced FET current of 20 μA. After mixing 10 μM of ligand with 30 μM of protein, the magnitude of ligand signal is reduced to 12 μA. For example, this means that the ligand signal is reduced by 40% (e.g., η=0.4), or 40% of the ligand molecules has reacted with the protein to form protein-ligand complex. Substituting the measured value of η into Eq. (7) and using the initial concentrations of ligand and protein molecules (e.g., [L]=10 μM and [P]=30 μM), obtained is the equilibrium coefficient:

$${K}_{eq} = \frac{1}{{10}^{- 5}} \frac{0.4}{\{ 3 - 0.4 \} \{ 1 - 0.4 \}} = 2.56 \times {10}^{- 4} {M}^{- 1} .$$

Following the same exemplary principles, one can obtain the protein-ligand reaction coefficients under different temperatures for first order and higher order reactions.

Measuring Protein-Ligand Reaction Rate

One can use the disclosed devices and the methods to measure the kinetics of the reaction as well. For example, using the exemplary device 160 and assuming a first order reaction for simplicity, the rate equations for the reaction in the micro chamber can be represented as

$$\begin{matrix}\frac{d [ L ( t ) ]}{d t} = - {K}_{->} [ L ( t ) ] [ P ( t ) ] + {K}_{←} [ LP ( t ) ] & ( 8 a ) \\ \frac{d [ P ( t ) ]}{d t} = - {K}_{->} [ L ( t ) ] [ P ( t ) ] + {K}_{←} [ LP ( t ) ] & ( 8 b ) \\ \frac{d [ LP ( t ) ]}{d t} = {K}_{->} [ L ( t ) ] [ P ( t ) ] - {K}_{←} [ LP ( t ) ] & ( 8 c )\end{matrix}$$

where K_{→} and K_{←} are the rate constants for the forward and reverse reactions to find out.

Although Eq. (8) is a nonlinear system with an analytical solution, one can set up the experimental conditions to have the initial ligand concentration much lower than the initial protein concentration (e.g., [L(0)]<<[P(0)]). Under such condition, one can assume that the following condition [P(t)]≅[P(0)] is always satisfied in Eq. (8). Using this approximation, one can solve the time dependent concentration for [L(t)] analytically

$$[ L ( t ) ] = \frac{[ L ( 0 ) ]}{{K}_{->} [ P ( 0 ) ] + {K}_{←}} \{ {K}_{←} + {K}_{->} [ P ( 0 ) ] {e}^{- ( {K}_{->} [ P ( 0 ) ] + {K}_{←} ) t} \} \qquad ( 9 )$$

where [L(0)] and [P(0)] are the initial concentration for ligand and protein.

To obtain the forward and reverse reaction coefficients K_{→} and K_{←}, the change of the ligand signal can be measured by waiting a certain period of time “T” before opening the microvalve, in some examples, between the molecular deposition chamber 110 and the microfluidic diffusion channel 120 of the exemplary device 160. Since the ligand molecule diffuses faster than the protein molecule and the protein-ligand complex, the ligand molecules at the leading edge of the diffusion profile soon outpace the protein and protein-ligand complex in the channel. These ligand molecules will have no protein molecules to react with. The ligand signals can be measured for different amounts of reaction times. When a long enough time duration has elapsed (T→∞) for the reaction to reach equilibrium, the following can be obtained:

$$\frac{[ L ( ∞ ) ]}{[ L ( 0 ) ]} = \frac{{K}_{←}}{{K}_{->} [ P ( 0 ) ] + {K}_{←}} \qquad ( 10 )$$

If one waits for a shorter time to open the valve before the equilibrium state is reached, the following can be obtained:

$$\frac{[ L ( T ) ]}{[ L ( 0 ) ]} = \frac{1}{{K}_{->} [ P ( 0 ) ] + {K}_{←}} \{ {K}_{←} + {K}_{->} [ P ( 0 ) ] {e}^{- ( {K}_{->} [ P ( 0 ) ] + {K}_{←} ) T} \} \qquad ( 11 )$$

Since one can measure

$$\frac{[ L ( ∞ ) ]}{[ L ( 0 ) ]} and \frac{[ L ( T ) ]}{[ L ( 0 ) ]}$$

from the ligand signals and know the initial concentration of protein [P(0)], K_{→}, and K_{←} can be solved from Eqs. (10) and (11).

Measuring Protein-Protein Binding

Besides measuring the binding of protein with small ligand molecules, the disclosed devices and methods can also be used to measure protein-protein binding. For example, if two proteins have similar sizes, one can detect their binding from the arrival time differences between the proteins and the protein-protein complex, as illustrated in FIG. 3B.

FIG. 3B shows a diagram of an exemplary molecular binding implementation using an exemplary device 100 of the disclosed technology to detect protein-protein interactions. FIG. 3B includes diagram 321 and 322 that show signals from the protein A alone and the protein B alone, respectively. FIG. 3B includes a diagram 323 that shows the signal from a protein/protein mix when binding does not occur. FIG. 3B includes a diagram 324 shows the signal from a protein/protein mixture when binding occurs.

In this example in FIG. 3B, protein A has a higher diffusivity than protein B, so two distinct current signals can be obtained at two different times (e.g., protein A at an earlier time than protein B, as depicted in the diagrams 321 and 322, respectively). If protein A and protein B can form a complex A-B, this molecule is expected to have a lower diffusivity than each individual protein and will arrive latest, as illustrated in the diagram 324 of FIG. 3B. By measuring the change of the signal magnitude of protein A and protein B due to the reaction, one can obtain both the reaction coefficient and the reaction rate in a similar manner to the case of protein-ligand interaction.

Using the same or similar concept and exemplary device structure, one can also investigate protein folding and reconfiguration under various influences such as gene mutations, drugs, ions (Mg⁺² , Ca⁺², etc.) and pH value. The underlying principle is that as protein molecules change their shape, the diffusivity or motion kinetics changes. By measuring the arrival time of proteins, one can detect protein configurations such as wild type proteins and genetically engineered proteins or proteins before and after post translational modifications. For example, to improve the temporal resolution, one can optimize the microfluidic channel geometry since the relative change of the diffusivity is proportional to the relative change of the diffusion time over the microfluidic channel:

$$\frac{Δ D}{D} = \frac{- Δτ}{τ} \qquad ( 12 )$$

The negative sign in Eq. (12) shows that an increase in the diffusivity causes a decrease in the diffusion time. The response time for the microvalve and the FET can each be in the order of 10 ms, so it is reasonable that one can precisely determine an arrival time difference of less than 1 second. In some exemplary implementations of the device 100 where the microfluidic channel includes a serpentine configuration (e.g., 70 μm deep, 100 μm wide, 0.5 mm long), which can give rise to a typical travel time of 4 minutes, the exemplary device has the sensitivity to detect a less than 1% change of the protein diffusivity. In other exemplary implementations of the device 100, the microfluidic channel is configured to be linear (e.g., 20-30 μm long, 30 μm deep, and 1 mm wide). In some exemplary implementations of the device 100 with variations of the FET sensing area and the channel geometry, as well as introduction of fine microscale or nanoscale structures, e.g., such as micro pillars, porous structures and hydrogel, a detection sensitivity of 0.1% diffusivity change can be detected the disclosed technology.

Detection of Charge Neutral Ligands and Proteins

So far, the previous description about protein-ligand and protein-protein interactions has been with charged molecules that could induce a current change of the FET when the molecules are in contact with the FET sensing area. The disclosed techniques can also be applied to charge neutral molecules. For example, before the detection implementation, the microfluidic channel can be filled with a salt medium in which protein and ligand molecules are suspended. In contact with the sensing area of the FET, the ions in the medium can change the FET current from its value in the air. This current can be used as the reference of the FET sensor without the presence of any protein or ligand molecules. When charge neutral molecules enter the FET sensing area, they may displace the ions and thus change the amount of the charge induced in the FET channel, thus producing a signal. The design works for charge neutral molecules that are polar or nonpolar through the change of dielectric constant by the presence of molecules within the Debye length. In Eq. (2), the surface charge density on the electrode depends on the dielectric constant of the solution. As the molecules for investigation approach the electrode, the dielectric properties of the medium change due to the “structure building” or “structure breaking” effect of the molecules on the microstructure of water. Furthermore, dielectric constant also appear in the expression of Debye length (e.g., Debye length is proportional to the square root of the dielectric constant according to the Debye-Hückel model), which affects the signal as well.

Also, for example, the exemplary device can work for other mediums, in addition to proteins in aqueous solution.

### Exemplary Embodiments

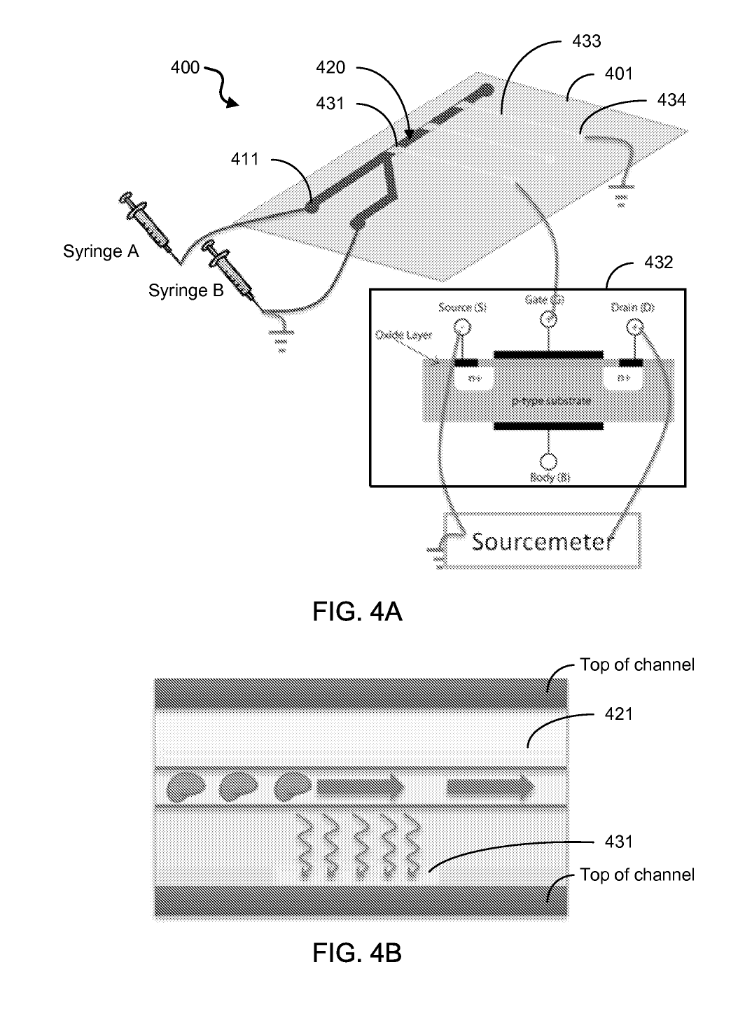

FIG. 4A shows a schematic diagram of one exemplary embodiment of the biomolecular interaction detection device 400. The device 400 includes a substrate 401 structured to form a microfluidic channel 420 having an array of electrodes 431 positioned along the length of the channel. In some implementations of the device 400, for example, the microfluidic channel 420 can be configured in the substrate 401 to have a 30 μm height and 1 mm width. For example, the electrodes 431 can be configured to be 1 mm×1mm (1 mm² area). The sensor module of the device 400 is configured such that the electrodes 431 of the array are electrically coupled to FETs 432 via interconnect wires 433, e.g., which can be embedded in the substrate 401. In some implementations, for example, the FETs 432 can be configured on the substrate 401, whereas in other implementations, for example, the FETs 432 can be included in an external electrical circuit that connects to the electrodes 431 by electrical connection to contact pads 434 via the interconnect wires 433. For example, the sensor module of the device 400 is configured to have the electrode 431 connected to the gate of the FET 432, e.g., an enhanced-mode (normally off) MOSFET operating in the subthreshold regime. For example, the FET 432 can also be configured as a nanoscale field-effect transistor, e.g., such as a JFET, MESFET, carbon nanotube FET, or nanowire FET, etc. The FETs 432 of the device 400 can be electrically connected to a source meter to monitor and display and/or output the detected signals. The substrate 401 is structured to form one or more fluid inlets 411. In some implementations, for example, the fluid inlets 411 can be connected to an external fluid delivery device, e.g., such as separate syringes containing different samples, shown as syringes A and B in FIG. 4A. In some implementations, for example, an electric ground can be connected to the syringe that introduces the molecules under investigation to avoid any statics that may cause artifacts in the signal.

Exemplary implementations using the device 400 were performed under various conditions and are described. In one implementation, for example, syringe A contained Tris buffer and syringe B contained 10 mM biotin in Tris buffer. The fluid channel was at first flown with Tris buffer from syringe A into the device 400 received at the corresponding inlet 411. At a particular time, the flow from syringe A was turned off and flow from syringe B was turned on, thus biotin-containing Tris buffer began entry to the microfluidic channel 420. Due to the nature of laminar flow in the microfluidic channel, the fluid in the center portion travels at a much greater velocity than the fluid near the channel wall. FIG. 4B shows a cross section view of the exemplary microfluidic channel 420 depicting the fluid distribution of the fluid 421 on top of the exemplary Au electrode 431 shortly after syringe B was turned on. The biotin-containing Tris was focused on the center of the channel surrounded by the Tris buffer originally from syringe A. For example, the disclosed technology creates the conditions to allow the measurement the diffusion properties of the biotin in Tris buffer.

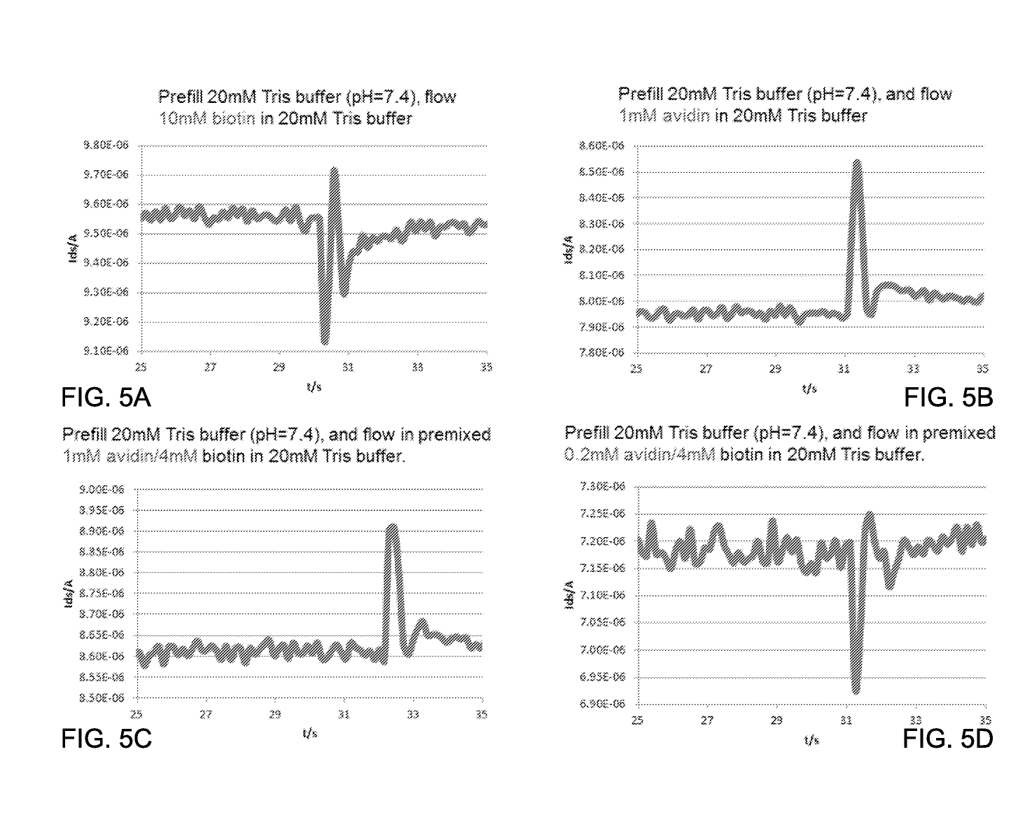

FIGS. 5A-5D show data plots of exemplary data measured at various conditions of the exemplary implementations to detect and analyze the protein avidin with the ligand biotin. The acquired signals were measured from the drain current of the exemplary FET 432. FIG. 5A shows a data plot displaying a transient signal with a characteristic waveform signifying the transport properties of biotin molecule in the Tris buffer. For example, 20 mM Tris buffer (pH=7.4) was prefilled in the channel and then 10 mM biotin was flowed in the 20 mM Tris buffer in the channel. This was similarly repeated with 1 mM streptavidin protein in the Tris buffer from syringe B. For example, 20 mM Tris buffer (pH=7.4) was prefilled in the channel and then 1 mM avidin was flowed in the 20 mM Tris buffer in the channel. FIG. 5B shows a data plot of a transient signal with a characteristic waveform signifying the transport properties of 1 mM streptavidin in the Tris buffer. Streptavidin is a protein that carries 20e positive charge for each molecule and has a diffusivity of 2.7×10⁻⁷ cm²/s, whereas biotin is a much smaller molecule as a ligand, carrying −e charge each and having a diffusivity of 3.4×10⁻⁶ cm²/s. The different waveform of the signal for biotin and streptavidin clearly indicates the different properties of these two molecules.

Next, the exemplary implementations included pre-mixing 1 mM of streptavidin with 4 mM of biotin in the Tris buffer before introducing it to the microfluidic channel 420, e.g., from syringe B. FIG. 5C shows a data plot displaying the measured signal of the pre-mixed 1 mM streptavidin −4 mM biotin sample. The signal shown in the diagram of FIG. 5C clearly resembles the streptavidin signal shown in FIG. 5B and is quite different from the biotin signal shown in FIG. 5A. This exemplary result indicates that streptavidin and biotin bind together. Since streptavidin (molecular weight: 66 KD) is much bigger than biotin (molecular weight: 0.244 KD) as a ligand molecule, it may be expected that its resulting signal is very similar to the signal of streptavidin (FIG. 5B) when biotin is bonded with streptavidin (FIG. 5C). For example, one streptavidin protein molecule can bind with 4 biotin molecules with a very high efficiency (e.g., a very low dissociation coefficient of K_{d}=0.6×10⁻¹⁵M).

Also, the exemplary implementations included changing the streptavidin to biotin ratio from 1:4 to 1:20, e.g., by reducing the streptavidin concentration from 1 mM to 0.2 mM in the Tris buffer while keeping the biotin concentration at 4 mM. FIG. 5D shows a data plot displaying the measured signal of the pre-mixed 0.2 mM streptavidin −4 mM biotin sample. It turns out that the signal carries the characteristics of both the signals shown in FIG. 5C and FIG. 5A, indicating that some biotin molecules have bonded with streptavidin to form the streptavidin/biotin complex but there exist extra biotin molecules that are not bonded to streptavidin. The exemplary waveform shown in FIG. 5D also shows that the characteristics of biotin appear before the characteristics of streptavidin/biotin complex because of biotin's greater diffusivity.

These exemplary implementations demonstrate the detection capability of protein-ligand binding (e.g., well-studied protein ligand molecules, streptavidin and biotin) using the disclosed technology in natural conditions without any labeling (e.g., without the use of fluorescent, FRET, quantum dot, etc.) and without any restriction of the degree of freedom of molecules (e.g., without the use of immobilization of the molecules to beads or solid surfaces).

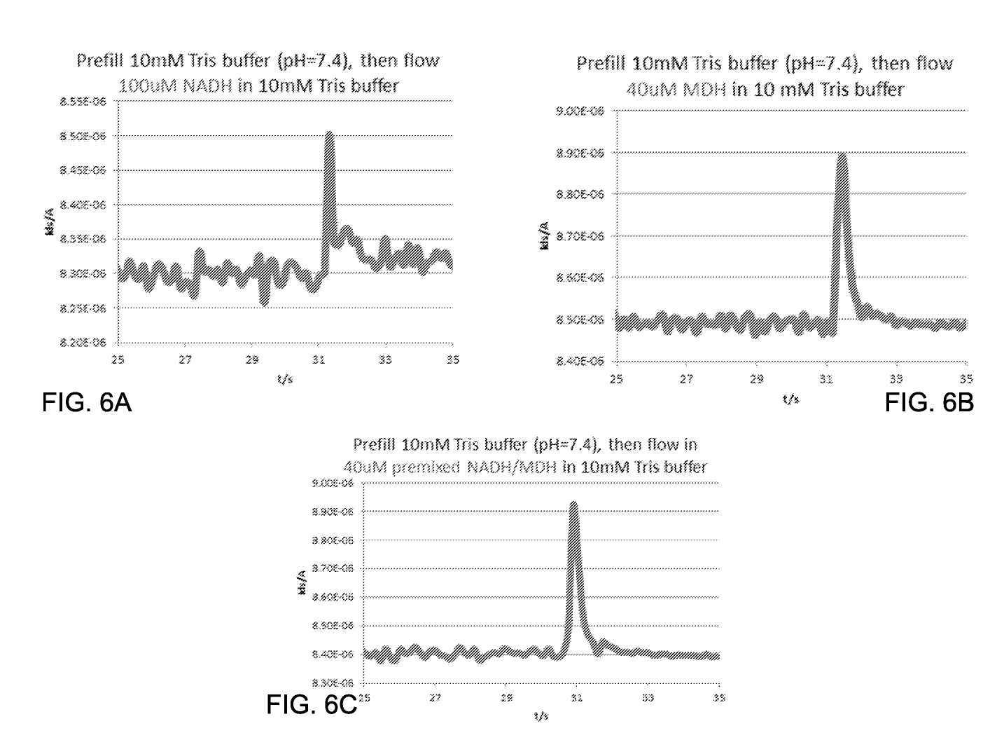

In another implementation using the device 400, for example, the binding between nicotinamide adenine dinucleotide (NADH) and malate dehydrogenase (MDH) protein was investigated. NADH is a relatively small molecule (0.644 KD) with a diffusivity of 2×10⁻⁶ cm²/s. MDH protein has its molecular weight of 33 KD and diffusivity of 4×10⁻⁷ cm²/s. The exemplary implementation included introducing NADH and MDH individually from syringe B in Tris buffer entry to the microfluidic channel 420. FIGS. 6A and 6B show data plots displaying transient signals with a characteristic waveform signifying the transport properties of NADH and MDH molecules in the Tris buffer, respectively. For example, 10 mM Tris buffer (pH=7.4) was prefilled in the channel and then 100 μM NADH was flowed in the 10 mM Tris buffer in the channel. Similarly, 40 μM MDH was flowed in the 10 mM Tris buffer in the channel. The pulse width of the NADH signal is narrower because of its higher diffusivity of the molecule.

Next, the exemplary implementations included pre-mixing an equal amount (e.g., 40 μM each) of NADH and MDH before being introduced to the microfluidic channel via syringe B. FIG. 6C shows a data plot displaying the measured signal of the pre-mixed 40 μM NADH-40 μM MDH sample. As shown in the diagram of FIG. 6C, the signal from the premixed sample possesses the characteristics of the MDH signal, indicating that NADH and MDH form protein/ligand duplex with the diffusivity similar to the value of MDH because of its much greater molecular weight.

The disclosed techniques of label-free, restriction-free protein-ligand detection and analysis can be implemented in a high-throughput platform, in which thousands of different molecules can be tested for their binding characteristics with target proteins.

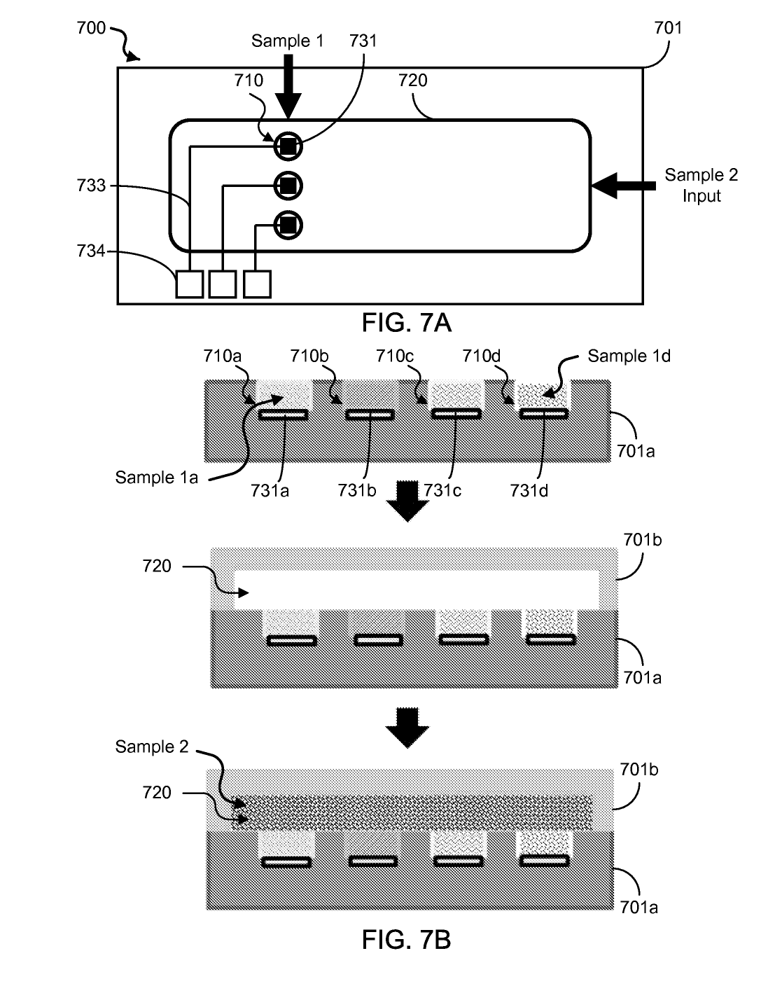

FIG. 7A shows a block diagram of an exemplary high-throughput biomolecular interaction detection device 700. The device 700 includes a substrate 701 structured to form an array of microwells 710 and a microfluidic channel 720 passing over the wells 710. The device 700 includes an array of electrodes 731 positioned in corresponding microwells of the array of microwells 710. In some implementations of the device 700, for example, the microfluidic channel 720 can be configured in the substrate 701 to have a particular height and width based on the arrangement and number of microwells 710 in the array, e.g., such as a 30 μm height and 10 mm width of the microfluidic channel 720. In some implementations of the device 700, for example, the microwells in the array can be configured in the substrate 701 to have a depth in a range of 20 to 50 μm deep and have a diameter in a range of 200 to 500 μm. For example, the microwells can be configured to have a cylindrical geometry, conical geometry, rectangular geometry, or other type of geometry formed in the substrate. In some implementations, for example, the electrodes 731 can be configured at the bottom of the microwells 710 of a metal (e.g., such as Au). Additionally or alternatively, for example, the electrodes 731 can be configured along a side of the microwells 710, or split into two parts sharing the same connection to the FET or being connected to separate FETs. The sensor module of the device 700 is configured such that the electrodes 731 of the array are electrically coupled to FETs via interconnect wires 733, e.g., which can be embedded in the substrate 701. For example, the FETs can be configured on the substrate 701 (not shown in FIG. 7A), or can be included in an external electrical circuit that connects to the electrodes 731 by electrical connection to contact pads 734 via the interconnect wires 733. For example, the sensor module of the device 700 can be configured to have the electrode 731 connected to the gate of the FET, e.g., an enhanced-mode (normally off) MOSFET operating in the subthreshold regime or a nanoscale field-effect transistor such as a JFET, MESFET, carbon nanotube FET, or nanowire FET, etc. The FETs of the device 700 can be electrically connected to a source meter to monitor and display and/or output the detected signals.

In some embodiments of the device 700, for example, the substrate 701 includes a lower substrate 701a and an upper substrate 701b that form the array of microwells 710 and the microfluidic channel 720. An example is depicted in FIG. 7B. FIG. 7B shows a schematic diagram of an exemplary method to prepare and implement the exemplary high-throughput device 700. The diagram of FIG. 7B shows a cross section of the device 700 depicting four microwells 710a, 710b, 710c, and 710d of the array 710 with four different fluid samples loaded in the respective microwells. In some implementations, the lower substrate 701a can be structured to form the microfluidic channel 720 with the array of microwells 710 formed within the formed channel 720. In such implementations, the upper substrate 701b can be used to seal the microfluidic channel 720 and underlying microwells 710 from being exposed. In other implementations, for example, the lower substrate 701a is structured to form the array of microwells 710, and the upper substrate 701b is structured to form the microfluidic channel 720, such that when the lower and upper substrates 701a and 701b are attached, the microfluidic channel 720 is aligned over the array of microwells 710, as depicted in FIG. 7B.

For example, one can use a robotically controlled or automatic spotting system to dispense fluid samples containing candidate ligands and/or proteins (e.g., sample 1) to investigate their interactions with a target biomolecule such as a protein (e.g., sample 2) in each microwell. For example, the fluid samples can be prepared to have a given amount of buffer with specific ligand candidate to deposit into the microwells. For example, the spotting process can be conducted in a high humidity environment or other controlled environment to prevent water evaporation from each microwell. After the large array of microwells 710 are loaded, the upper substrate 701b (e.g., a cap) is placed over the lower substrate 701b. In such exemplary implementations, the space between the wells and the overlaying cap forms the microfluidic channel 720. The buffer solution containing the target protein is then introduced to the microfluidic channel 720. The liquid interface is formed between the target protein-containing buffer and the candidate ligand-loaded solution in the microwells, and the diffusion process can begin. For example, as the candidate ligands diffuse out of the microwells and the target protein diffuses into each microwell, a detectable signal is produced for each microwell as a result of the ion current and the induced surface charge on the electrode in each microwell. If binding between protein and ligand occurs, the out-diffused ligand molecule is brought back into the microwell, carried by the protein of greater mass. In this manner, the device 700 can detect the presence (or absence) of protein-ligand binding as well as the binding kinetics from the waveform of the signal from each microwell.

The design of the device 700 produces negligible or minimal crosstalk or interference, as the trace amount of ligand molecules diffused out of each microwell leaves the device quickly and its concentration is orders of magnitude below that of protein in the flow.

In one exemplary embodiment of the biomolecular interaction detection device, for example, the sensing module can include thin film transistor (TFT) technology (e.g., like that for flat panel displays) used as the sensing field-effect transistor (FET), and the FET is integrated with a microfluidic channel with or without microfluidic valves. This exemplary embodiment is cost effective and easy to scale to support thousands of protein-ligand binding tests in parallel for high-throughput drug screening. The TFT technology can enable low production cost of such devices, and thereby allow the devices to be disposable and in turn minimize cross contamination and fouling.

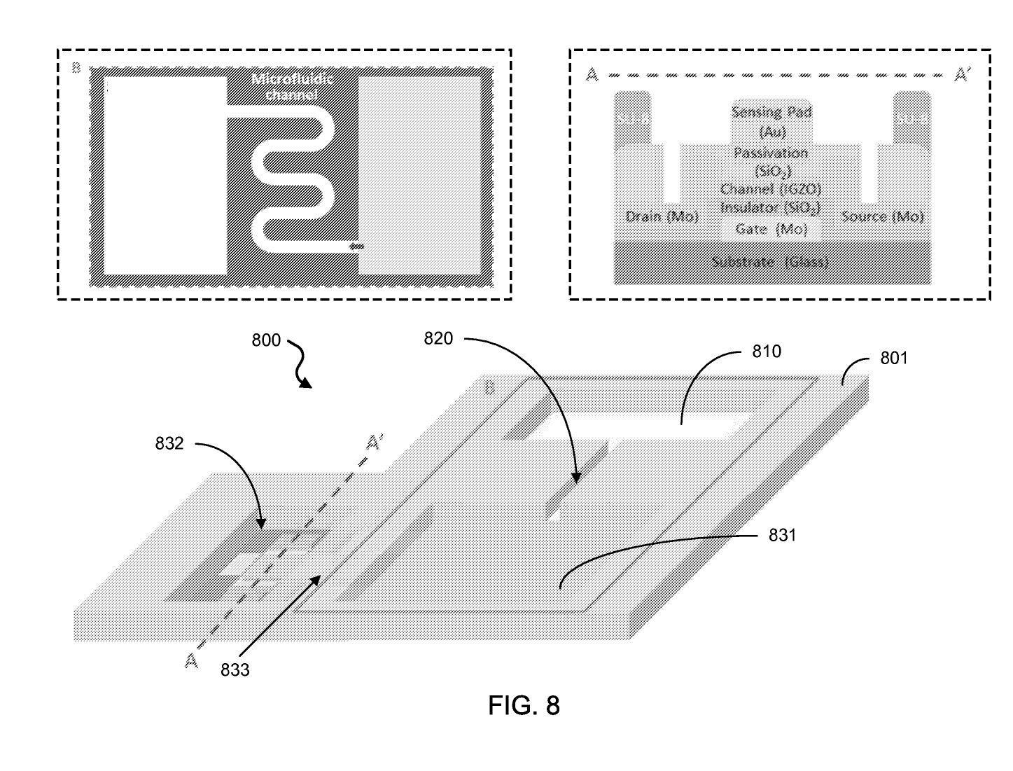

FIG. 8 shows a schematic diagram of an exemplary biomolecular interaction detection device 800 including TFTs in the sensor module. The device 800 includes a substrate 801 structured to form a molecular deposition chamber 810 and a microfluidic channel 820 having one or more electrodes 831 that can be positioned along the length of the channel or at an end of the channel. In some implementations of the device 800, for example, the microfluidic channel 820 can be configured in the substrate 801 to have a 30 μm height and 1 mm width and have a particular geometry, e.g., such as a linear or serpentine geometry, etc. In some implementations, the electrodes 831 can be configured as 1 mm² area electrodes in the channel, whereas in other implementations, for example, a single electrode pad can be configured at an opposite end of the microfluidic channel 820 than that of the molecular deposition chamber 810, as depicted in FIG. 8. The sensor module of the device 800 is configured such that the electrode(s) 831 are electrically coupled to corresponding FET(s) 832 via interconnect wires 833, e.g., which can be embedded in the substrate 801. In some implementations of the device 800, for example, the FET(s) 832 can be included in an external electrical circuit that connects to the electrode(s) 831 by electrical connection to contact pads (not shown) via the interconnect wires 833. For example, the FET(s) 832 of the device 800 can be electrically connected to a source meter to monitor and display and/or output the detected signals. Insets at the top of the diagram of FIG. 8 show a vertical cross section A to A′ of the exemplary FET of the device 800 and a horizontal cross sectional area B of the exemplary deposition chamber, an exemplary serpentine microfluidic channel, and an exemplary extended electrode pad of the device 800.

In some implementations, for example, the device 800 can include Indium-Gallium-Zinc Oxide (IGZO) TFT FETs 832 coupled to the electrodes 831 configured along or at one end of the microfluidic channel 820. For example, to function as a sensing transistor, a TFT with an extended sensing metal pad can be fabricated on the substrate 801, e.g., glass substrate. The exemplary extended metal electrode pad 831 can be used to sense the induced charge on the pad. It is electrically connected to the gate of the exemplary IGZO TFT FET 832, but fluidically isolated from the rest of the TFT to avoid electric leakage and hydrolysis. In addition to IGZO TFT-FET, for example, the sensing device can also be made of amorphomous silicon TFT, low temperature polysilicon TFT, and other technologies.

An exemplary fabrication method is described that can be implemented to produce the exemplary biomolecular interaction detection device 800 shown in FIG. 8. The exemplary TFT has two gates that sandwich the IGZO channel. In some examples, the staggered bottom-gate can be fabricated on a Corning Eagle 2000 glass substrate. The exemplary method can include, for example, DC sputtering of a 150 nm-thick Mo thin film followed by reactive ion etch (RIE). Then an 80 nm-thick SiO₂ dielectric layer can be deposited by plasma enhanced chemical vapor deposition (PECVD). To form the channel of the TFT-FET, for example, a 50 nm IGZO film is coated by RF sputtering at room temperature, and the channel mesa is patterned by wet etch with diluted HCl. The source and drain contacts can be formed with Mo metal that is DC sputtered and patterned by RIE. To reduce the RIE induced defects on the surface of IGZO film, for example, the IGZO film is dipped in diluted HCl again for a few seconds after the formation of Mo source and drain contacts. To protect the devices and to form an insulating layer for the sensing metal pad connected to the top gate of the TFT-FET, for example, a 100 nm thick SiO₂ layer is deposited by RF sputtering at room temperature. Via-holes are opened to expose the contact pads for the source, drain, and bottom gate.

To fabricate the top gate of the TFT-FET as the sensing pad, for example, a 300 nm gold pad can be formed by E-beam evaporation and lift-off process. For example, gold can be selected as the material of the sensing pad because of its biocompatibility and wide usage in biomedical devices. The TFT-FET can be annealed at 300° C. under nitrogen ambient for an hour to attain good electrical properties. To integrate microfluidic structures with the TFT-FET, for example, a 100 μm thick SU-8 photoresist can be coated and patterned by photolithography to form the microfluidic channels and reservoirs, as shown in FIG. 8. If desired, additional processes can be applied to form microfluidic valves to control the fluid exchanges between the reservoir 810 at the inlet and the microfluidic channel 820. To facilitate fluid fill and remove any trapped air bubbles before test, another reservoir at the outlet may be added to the device 800. The device 800 can be fabricated in large volume at low cost.

Exemplary implementations were performed using the device 800 to demonstrate aspects of device functionality The exemplary implementations included filling the sensing pad area and the microfluidic channel with phosphate buffered saline (PBS) before introducing an exemplary protein (e.g., IgG antibody) solution to the inlet. The IgG antibody was diffused from the pool to the sensing pad through a microfluidic channel because of the concentration gradient. The change of the TFT drain current occurs as soon as the IgG antibody reaches the electrode pad 831. In some implementations, for example, the exemplary IGZO channel can be configured to be 600 μm wide and 250 μm long and can be biased at V_{GS}=8 V and V_{DS}=5V.

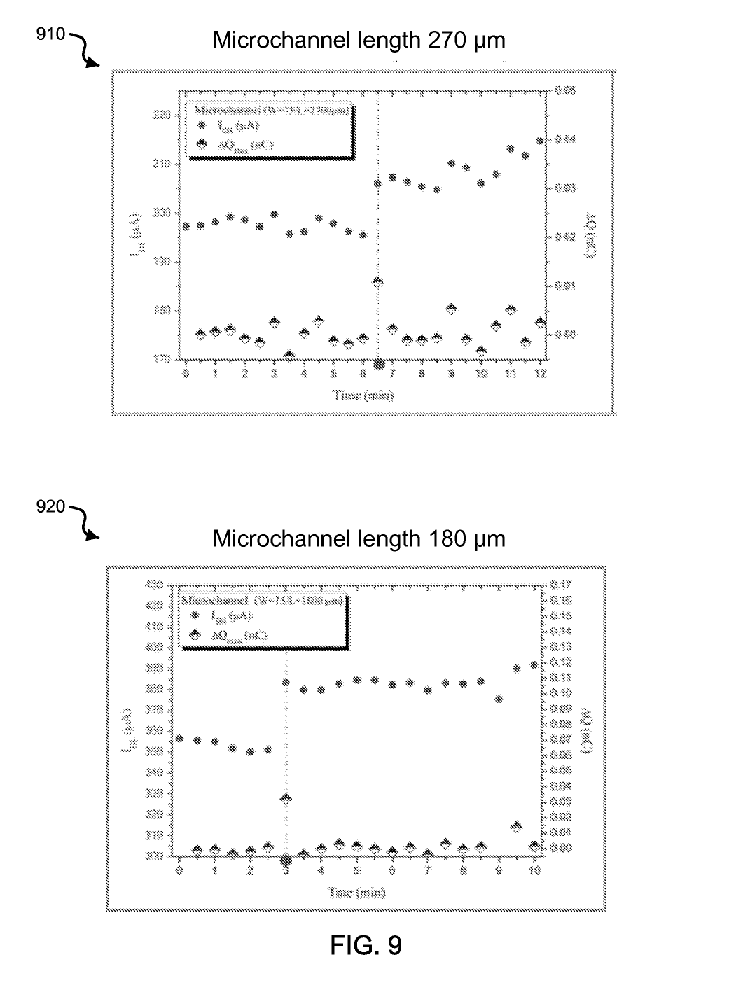

In the exemplary implementations of the device 800 including the microfluidic channel 820 having a serpentine configuration and length of 270 μm, the drain current can suddenly be increased from 195.8 to 206.1 μA at 6.5 minutes after the introduction of IgG antibody, e.g., indicating that in 6.5 minutes the IgG has reached the sensing pad. FIG. 9 shows data plots 910 and 920 showing exemplary TFT signals for protein detection for two exemplary devices having different microfluidic channel lengths, e.g., 270 μm and 180 μm. Table 1 presents these exemplary results.

TABLE 1

| Microchannel length (μm) | Response time (min) | Drain current (μA) |
|---|---|---|
| 270 | 6.5 | 195.8 → 206.1 |
| 180 | 3.0 | 350.5 → 383.6 |

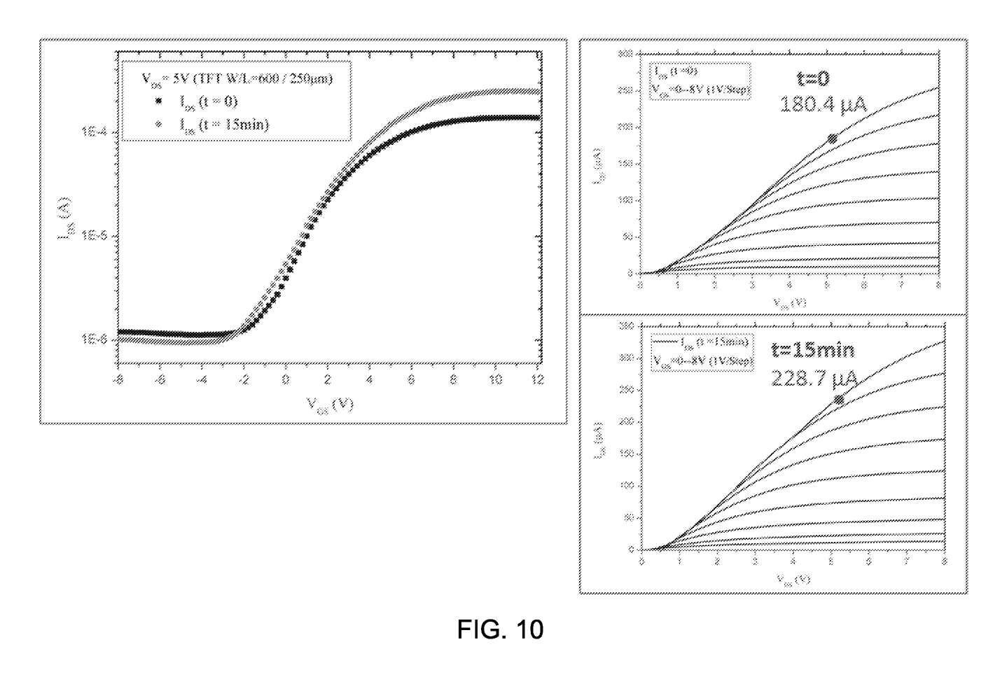

FIG. 10 shows data plots depicting the drain current variation by molecules in the fluid, e.g., showing the exemplary I-V characteristics of the TFT modulated by the IgG antibody. The exemplary results show that IgG antibody produces little changes of the threshold voltage and the subthreshold characteristics of the exemplary device 800, indicating that the intrinsic channel property of the TFT is not affected by the test.

A similar test was also performed with an exemplary device 800 having a shorter 180 μm microfluidic channel length. At 3 minutes after the introduction of IgG to the channel, a sudden increase of current from 350.5 to 383.6 μA (as shown in the diagram 920 of FIG. 9) was detected.

The disclosed systems, devices, and techniques include a device architecture and a methodology to enable investigation of protein-ligand and protein-protein interactions as well as fundamental protein properties in conditions close to the physiological environments. The exemplary techniques require no labeling of the molecules, and impose no constraints on the motions of the molecules under study. Exemplary results obtained from exemplary implementations of exemplary devices and techniques of the disclosed technology provided results to be closest to the in vivo results. In some examples, an exemplary technique of the disclosed technology can be implemented to produce both qualitative (e.g., whether ligand-protein binding occurs or not) and quantitative (e.g., the reaction constants) information, and is applicable to a large variety of proteins and ligands of different molecular weight, charge, hydrophobicity, and 3D configurations. Applications of the disclosed technology can include applications in high-throughput drug screening and biological sciences, among others.

### EXAMPLES

The following examples are illustrative of several embodiments of the present technology. Other exemplary embodiments of the present technology may be presented prior to the following listed examples, or after the following listed examples.

In an example of the present technology (example 1), a high-throughput molecular interaction detection device includes a substrate including an electrically insulative material and structured to form (i) an array of wells to receive corresponding fluid samples including candidate molecules, and (ii) a microfluidic channel positioned above openings of the wells, in which the microfluidic channel is shaped to carry a fluid including target biomolecules to the openings of the wells to create fluid interfaces between the fluid and the fluid samples; an electrode disposed on a surface of each well to detect a change in an electric signal based at least partly on molecular interactions between the target biomolecules and candidate molecules in a respective well; and a plurality of transistors electrically coupled to corresponding electrodes to generate an output signal based at least partly on the detected change in the electrical signal.

Example 2 includes the device as in example 1, in which the array of wells and the microfluidic channel are arranged on the substrate to enable the candidate molecules and the target biomolecules to diffuse across the fluid interfaces to enter and exit respective wells at different diffusivities, respectively, such that: a given molecular interaction between a given target biomolecule and a given candidate molecule induces a surface charge on the corresponding electrode to change the electrical signal detected by the corresponding electrode; and at least some of the diffusion transported candidate molecules interact with at least some of the target biomolecules outside the respective well.

Example 3 includes the device as in example 2, in which the electrode is disposed on the surface of each well to detect the change in the electric signal based at least partly on the molecular interactions including binding of the candidate molecules to the target biomolecules.

Example 4 includes the device as in example 3, in which the array of wells and the microfluidic channel are arranged on the substrate to enable the at least some of the candidate molecules that bind with the at least some of the target biomolecules outside the respective wells to be brought back into respective wells attached to the bound target biomolecules.

Example 5 includes the device as in example 1, in which the substrate includes an upper substrate and a lower substrate, in which the lower substrate is structured to form the microfluidic channel and the array of wells arranged within the formed microfluidic channel, and the upper substrate is configured on top of the lower substrate to enclose the microfluidic channel and the array of wells.

Example 6 includes the device as in example 1, in which the substrate includes an upper substrate and a lower substrate, in which the lower substrate is structured to form the array of wells, and the upper substrate is structured to form the microfluidic channel and configured to attach to the lower substrate, such that when the lower and upper substrates and are attached, the microfluidic channel is aligned over the array of wells.

Example 7 includes the device as in example 1, in which at least some of the candidate molecules and the target biomolecules are not labeled and are not immobilized to the substrate.

Example 8 includes the device as in example 1, in which the array of wells includes at least a hundred wells.

Example 9 includes the device as in example 1, in which the candidate molecules and the target biomolecules include at least one of proteins or ligands.

Example 10 includes the device as in example 1, in which the target biomolecules include proteins and the candidate molecules include drugs.

Example 11 includes the device as in example 1, further including electrical interconnect wires embedded in the substrate to electrically connect the transistors to the corresponding electrodes, in which the transistors are embedded in or attached on the substrate.

Example 12 includes the device as in example 1, in which the transistors are included in an external electrical circuit, and the device further includes contact pads formed of an electrically conductive material on the substrate and capable of electrically connecting to the external electrical circuit; and electrical interconnect wires embedded in the substrate to electrically connect the contact pads to the corresponding electrodes.

Example 13 includes the device as in example 1, in which the transistor includes a metal-oxide-semiconductor field effect transistor (MOSFET).

Example 14 includes the device as in example 1, in which the wells of the array are configured to have a depth in a range of 20 to 50 μm and a diameter in a range of 200 to 500 μm.

Example 15 includes the device as in example 1, in which the microfluidic channel is configured to be a linear channel having a length in a range of 20 to 30 μm.

In an example of the present technology (example 16), a device to detect molecular interactions includes a substrate including an electrically insulative material and structured to form a microfluidic channel to receive one or more fluid samples including biomolecules at a first region of the channel and to carry the fluid to a second region of the channel, in which the microfluidic channel is arranged on the substrate to enable a given biomolecule to undergo a molecular interaction with another given biomolecule that alters a molecular property of one or both the given biomolecule and the other given biomolecule to become a molecular-interacted biomolecule; an electrode disposed on a surface of the microfluidic channel in the second region to detect a change in an electrical signal based at least partly on molecular interactions of the biomolecules; and a transistor electrically coupled to the electrode to generate an output signal based at least partly on the detected change in the electrical signal.

Example 17 includes the device as in example 16, in which the biomolecules include one or both of proteins and ligands.

Example 18 includes the device as in example 17, in which the molecular interaction includes a protein-ligand binding or a protein-protein binding.

Example 19 includes the device as in example 17, in which the changed molecular property includes protein folding or conformational change, protein denaturing, or protein surface charge alteration.

Example 20 includes the device as in example 16, in which the biomolecules are not labeled and are not immobilized to the substrate.

Example 21 includes the device as in example 16, in which the substrate is structured to form a molecular deposition chamber at the first region of the microfluidic channel to receive two or more fluid samples each including different biomolecules, in which the molecular deposition chamber structured to enable the biomolecules to undergo the molecular interactions in the molecular deposition chamber.

Example 22 includes the device as in example 21, further including a microscale valve configured between the molecular deposition chamber and the microfluidic channel of the substrate, in which the microscale valve is structured to contain the biomolecules in the molecular deposition chamber and open to allow the biomolecules diffuse into the microfluidic channel.

Example 23 includes the device as in example 16, further including electrical interconnect wires embedded in the substrate to electrically connect the transistor to the electrode, in which the transistor is embedded in or attached on the substrate.

Example 24 includes the device as in example 16, in which the transistor is included in an external electrical circuit, and the device further includes a contact pad formed of an electrically conductive material on the substrate and capable of electrically connecting to the external electrical circuit; and an electrical interconnect wire embedded in the substrate to electrically connect the contact pad to the electrode.

Example 25 includes the device as in example 16, in which the transistor includes a metal-oxide-semiconductor field effect transistor (MOSFET) or a thin film field effect transistor (TF-FET).

Example 26 includes the device as in example 16, in which the microfluidic channel is configured to be a linear channel having a length in a range of 20 to 30 μm.

Example 27 includes the device as in example 16, in which the microfluidic channel is configured to be a serpentine channel having a length in a range of 1 to 2 mm.

Example 28 includes the device as in example 16, in which the electrode includes a surface functionalized or patterned metal.

Example 29 includes the device as in example 16, in which the first region of the microfluidic channel includes an plurality of subchannels that branch from the microfluidic channel to receive a corresponding fluid sample including different biomolecules with respect to another fluid sample.

Example 30 includes the device as in example 16, in which the substrate includes an upper substrate and a lower substrate, in which the lower substrate is structured to form the microfluidic channel, and the upper substrate is configured on top of the lower substrate to enclose the microfluidic channel.

In an example of the present technology (example 31), a device to detect molecular interactions includes a substrate formed of an electrically insulative material, in which the substrate is structured to form (i) a molecular deposition chamber to receive one or more fluid samples including biomolecules, in which the biomolecules are capable of undergoing molecular interactions in the molecular deposition chamber that changes a molecular property of the molecular-interacted biomolecules, and (ii) a microfluidic channel to carry the biomolecules, in which, based at least partly on the molecular interactions, the biomolecules travel through the microfluidic channel with different diffusivities; and an electronic sensor including an electrode configured along or at one end of the microfluidic channel and a transistor to detect the changed molecular property of the molecular-interacted biomolecules as a change in electrical signal, in which the electronic sensor is operable to produce an output signal corresponding to the detected electrical signal.

Example 32 includes the device as in example 31, in which the biomolecules include at least one of proteins or ligands.

Example 33 includes the device as in example 31, in which the changed molecular property is a result of a protein-ligand binding, protein-protein interaction, protein folding or reconfiguration detection, or a molecular denaturing, charge, or diffusivity.

Example 34 includes the device as in example 31, in which the detected change in electrical signal is based at least partly on different times of arrivals at the electrode of the molecular-interacted biomolecules.

Example 35 includes the device as in example 31, in which the electrical signal change is at least one of a change in current or voltage.

Example 36 includes the device as in example 31, in which the transistor of the electronic sensor includes a thin film field effect transistor (TF-FET).

Example 37 includes the device as in example 36, in which the TF-FET is embedded in the substrate.

Example 38 includes the device as in example 37, in which the TF-FET structured to include at least a part of its gate area electrically coupled to the electrode configured in the microfluidic channel.

Example 39 includes the device as in example 31, in which the electrode includes a surface functionalized or patterned metal.

Example 40 includes the device as in example 31, further including a microscale valve configured between the molecular deposition chamber and the microfluidic channel of the substrate, in which the microscale valve is structured to contain the biomolecules in the molecular deposition chamber and open to allow the biomolecules diffuse into the microfluidic channel.

In an example of the present technology (example 41), a method to detect molecular interactions includes receiving a fluid sample including biomolecules in a microfluidic channel at a first region of the microfluidic channel to flow the fluid sample carrying the biomolecules through the microfluidic channel to a second region of the channel; detecting a change in an electrical signal at an electrode disposed on a surface of the microfluidic channel in the second region, in which the detected change in the electrical signal is based at least partly on molecular interactions among the biomolecules causing an induced surface charge on the electrode; and processing the detected change in the electrical signal to determine an occurrence of the molecular interactions among the biomolecules.

Example 42 includes the method as in example 41, in which the processing the detected electrical signal includes acquiring an output signal from a transistor electrically coupled to the electrode.

Example 43 includes the method as in example 41, in which the biomolecules include one or both of proteins and ligands.

Example 44 includes the method as in example 43, in which the molecular interactions include at least one of protein-ligand binding or protein-protein interaction.

Example 45 includes the method as in example 43, in which the molecular interactions among the biomolecules alters a molecular property of at least one of the molecular-interacted biomolecules, in which the changed molecular property includes at least one of protein folding or conformational change, protein denaturing, or protein surface charge alteration.

Example 46 includes the method as in example 41, in which the biomolecules are not labeled and are not immobilized to a surface in the microfluidic channel.

Example 47 includes the method as in example 41, in which the receiving the fluid sample includes sequentially receiving a first fluid sample including a first type of biomolecules and a second fluid sample including a second type of biomolecules having a slower diffusivity than the first type, in which the processing includes determining the occurrence of molecular interactions between the first and second types of biomolecules when the change in the electrical signal includes an amplitude increase of a waveform of the first type of biomolecules.

In an example of the present technology (example 48), a method for high-throughput detection of molecular interactions includes receiving a plurality of fluid samples including candidate molecules in an array of wells formed on a substrate; receiving a fluid including target biomolecules in a microfluidic channel formed on the substrate in fluidic connection with the array of wells, in which the fluid carrying the target biomolecules from the microfluidic channel to openings of the wells create fluid interfaces between the fluid and the fluid samples; detecting a change in an electrical signal from an electrode disposed on a surface of a corresponding well, in which the detected change in the electrical signal is based at least partly on molecular interactions between the target biomolecules and candidate molecules causing an induced surface charge on the corresponding electrode; and processing the detected change in the electrical signal from each electrodes associated to the corresponding wells to determine an occurrence of the molecular interactions between the target biomolecules and the respective candidate molecules.

Example 49 includes the method as in example 48, in which the receiving the fluidic samples in the array of wells and the receiving the fluid in the microfluidic channel enable the candidate molecules and the target molecules, respectively, to diffuse across the fluid interface from the corresponding wells with different diffusivities such that: a given molecular interaction between a given target biomolecule and a given candidate molecule induces a surface charge on the corresponding electrode to change the electrical signal detected at the corresponding electrode; and at least some of the diffusion transported candidate molecules interact with at least some of the target biomolecules proximate to or in the corresponding well.

Example 50 includes the method as in example 49, in which the molecular interactions between the target biomolecules and the respective candidate molecules in or out of the corresponding well include binding of the candidate molecule to the target biomolecule.

Example 51 includes the method as in example 50, in which the binding of the candidate molecules to the target biomolecules out of the corresponding well results in candidate molecules being brought back into their respective well attached to the bound target biomolecule.

Example 52 includes the method as in example 48, in which at least some of the candidate molecules and the target biomolecules are not labeled and are not immobilized to the substrate.

Example 53 includes the method as in example 48, in which the array of wells includes at least a hundred wells.

Example 54 includes the method as in example 48, in which the candidate molecules and the target biomolecules include at least one of proteins or ligands.

Example 55 includes the method as in example 48, in which the target biomolecules include proteins and the candidate molecules include drugs.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A high-throughput molecular interaction detection device, comprising:
a substrate including an electrically insulative material and structured to form (i) an array of wells to receive corresponding fluid samples including candidate molecules, and (ii) a microfluidic channel positioned above openings of the wells, wherein the microfluidic channel is shaped to carry a fluid including target biomolecules to the openings of the wells to create fluid interfaces between the fluid and the fluid samples;
an electrode disposed on a surface of each well to detect a change in an electric signal based at least partly on molecular interactions between the target biomolecules and candidate molecules in a respective well; and
a plurality of transistors electrically coupled to corresponding electrodes to generate an output signal based at least partly on the detected change in the electrical signal.

2. The device as in claim 1, wherein the array of wells and the microfluidic channel are arranged on the substrate to enable the candidate molecules and the target biomolecules to diffuse across the fluid interfaces to enter and exit respective wells at different diffusivities, respectively, such that:
a given molecular interaction between a given target biomolecule and a given candidate molecule induces a surface charge on the corresponding electrode to change the electrical signal detected by the corresponding electrode; and
at least some of the diffusion transported candidate molecules interact with at least some of the target biomolecules outside the respective well.

3. The device as in claim 2, wherein the electrode is disposed on the surface of each well to detect the change in the electric signal based at least partly on the molecular interactions including binding of the candidate molecules to the target biomolecules.

4. The device as in claim 3, wherein the array of wells and the microfluidic channel are arranged on the substrate to enable the at least some of the candidate molecules that bind with the at least some of the target biomolecules outside the respective wells to be brought back into respective wells attached to the bound target biomolecules.

5. The device as in claim 1, wherein the substrate includes an upper substrate and a lower substrate, wherein the lower substrate is structured to form the microfluidic channel and the array of wells arranged within the formed microfluidic channel, and the upper substrate is configured on top of the lower substrate to enclose the microfluidic channel and the array of wells.

6. The device as in claim 1, wherein the substrate includes an upper substrate and a lower substrate, wherein the lower substrate is structured to form the array of wells, and the upper substrate is structured to form the microfluidic channel and configured to attach to the lower substrate, such that when the lower and upper substrates and are attached, the microfluidic channel is aligned over the array of wells.

7. The device as in claim 1, wherein at least some of the candidate molecules and the target biomolecules are not labeled and are not immobilized to the substrate.

8. The device as in claim 1, wherein the array of wells includes at least a hundred wells.

9. The device as in claim 1, wherein the candidate molecules and the target biomolecules include at least one of proteins or ligands.

10. The device as in claim 1, wherein the target biomolecules include proteins and the candidate molecules include drugs.

11. The device as in claim 1, further comprising:
electrical interconnect wires embedded in the substrate to electrically connect the transistors to the corresponding electrodes,
wherein the transistors are embedded in or attached on the substrate.

12. The device as in claim 1, wherein the transistors are included in an external electrical circuit, and the device further comprises:
contact pads formed of an electrically conductive material on the substrate and capable of electrically connecting to the external electrical circuit; and
electrical interconnect wires embedded in the substrate to electrically connect the contact pads to the corresponding electrodes.

13. The device as in claim 1, wherein the transistor includes a metal-oxide-semiconductor field effect transistor (MOSFET).

14. The device as in claim 1, wherein the wells of the array are configured to have a depth in a range of 20 to 50 μm and a diameter in a range of 200 to 500 μm.

15. The device as in claim 1, wherein the microfluidic channel is configured to be a linear channel having a length in a range of 20 to 30 μm.

16. A device to detect molecular interactions, comprising:
a substrate including an electrically insulative material and structured to form a microfluidic channel to receive one or more fluid samples including biomolecules at a first region of the channel and to carry the fluid to a second region of the channel, wherein the microfluidic channel is arranged on the substrate to enable a given biomolecule to undergo a molecular interaction with another given biomolecule that alters a molecular property of one or both the given biomolecule and the other given biomolecule to become a molecular-interacted biomolecule;
an electrode disposed on a surface of the microfluidic channel in the second region to detect a change in an electrical signal based at least partly on molecular interactions of the biomolecules; and
a transistor electrically coupled to the electrode to generate an output signal based at least partly on the detected change in the electrical signal.

17. The device as in claim 16, wherein the biomolecules include one or both of proteins and ligands.

18. The device as in claim 17, wherein the molecular interaction includes a protein-ligand binding or a protein-protein binding.

19. The device as in claim 17, wherein the changed molecular property includes protein folding or conformational change, protein denaturing, or protein surface charge alteration.

20. The device as in claim 16, wherein the biomolecules are not labeled and are not immobilized to the substrate.

21. The device as in claim 16, wherein the substrate is structured to form a molecular deposition chamber at the first region of the microfluidic channel to receive two or more fluid samples each including different biomolecules, wherein the molecular deposition chamber structured to enable the biomolecules to undergo the molecular interactions in the molecular deposition chamber.

22. The device as in claim 21, further comprising:
a microscale valve configured between the molecular deposition chamber and the microfluidic channel of the substrate,
wherein the microscale valve is structured to contain the biomolecules in the molecular deposition chamber and open to allow the biomolecules diffuse into the microfluidic channel.

23. The device as in claim 16, further comprising:
electrical interconnect wires embedded in the substrate to electrically connect the transistor to the electrode,
wherein the transistor is embedded in or attached on the substrate.

24. The device as in claim 16, wherein the transistor is included in an external electrical circuit, and the device further comprises:
a contact pad formed of an electrically conductive material on the substrate and capable of electrically connecting to the external electrical circuit; and
an electrical interconnect wire embedded in the substrate to electrically connect the contact pad to the electrode.

25. The device as in claim 16, wherein the transistor includes a metal-oxide-semiconductor field effect transistor (MOSFET) or a thin film field effect transistor (TF-FET).

26. The device as in claim 16, wherein the microfluidic channel is configured to be a linear channel having a length in a range of 20 to 30 μm.

27. The device as in claim 16, wherein the microfluidic channel is configured to be a serpentine channel having a length in a range of 1 to 2 mm.

28. The device as in claim 16, wherein the electrode includes a surface functionalized or patterned metal.

29. The device as in claim 16, wherein the first region of the microfluidic channel includes an plurality of subchannels that branch from the microfluidic channel to receive a corresponding fluid sample including different biomolecules with respect to another fluid sample.

30. The device as in claim 16, wherein the substrate includes an upper substrate and a lower substrate, wherein the lower substrate is structured to form the microfluidic channel, and the upper substrate is configured on top of the lower substrate to enclose the microfluidic channel.

31. A device to detect molecular interactions, comprising:
a substrate formed of an electrically insulative material, wherein the substrate is structured to form (i) a molecular deposition chamber to receive one or more fluid samples including biomolecules, wherein the biomolecules are capable of undergoing molecular interactions in the molecular deposition chamber that changes a molecular property of the molecular-interacted biomolecules, and (ii) a microfluidic channel to carry the biomolecules, wherein, based at least partly on the molecular interactions, the biomolecules travel through the microfluidic channel with different diffusivities; and
an electronic sensor including an electrode configured along or at one end of the microfluidic channel and a transistor to detect the changed molecular property of the molecular-interacted biomolecules as a change in electrical signal, wherein the electronic sensor is operable to produce an output signal corresponding to the detected electrical signal.

32. The device as in claim 31, wherein the biomolecules include at least one of proteins or ligands.

33. The device as in claim 31, wherein the changed molecular property is a result of a protein-ligand binding, protein-protein interaction, protein folding or reconfiguration detection, or a molecular denaturing, charge, or diffusivity.

34. The device as in claim 31, wherein the detected change in electrical signal is based at least partly on different times of arrivals at the electrode of the molecular-interacted biomolecules.

35. The device as in claim 31, wherein the electrical signal change is at least one of a change in current or voltage.

36. The device as in claim 31, wherein the transistor of the electronic sensor includes a thin film field effect transistor (TF-FET).

37. The device as in claim 36, wherein the TF-FET is embedded in the substrate.

38. The device as in claim 37, wherein the TF-FET structured to include at least a part of its gate area electrically coupled to the electrode configured in the microfluidic channel.

39. The device as in claim 31, wherein the electrode includes a surface functionalized or patterned metal.

40. The device as in claim 31, further comprising:
a microscale valve configured between the molecular deposition chamber and the microfluidic channel of the substrate,
wherein the microscale valve is structured to contain the biomolecules in the molecular deposition chamber and open to allow the biomolecules diffuse into the microfluidic channel.

41. A method to detect molecular interactions, comprising:
receiving a fluid sample including biomolecules in a microfluidic channel at a first region of the microfluidic channel to flow the fluid sample carrying the biomolecules through the microfluidic channel to a second region of the channel;
detecting a change in an electrical signal at an electrode disposed on a surface of the microfluidic channel in the second region, wherein the detected change in the electrical signal is based at least partly on molecular interactions among the biomolecules causing an induced surface charge on the electrode; and
processing the detected change in the electrical signal to determine an occurrence of the molecular interactions among the biomolecules.

42. The method as in claim 41, wherein the processing the detected electrical signal includes acquiring an output signal from a transistor electrically coupled to the electrode.

43. The method as in claim 41, wherein the biomolecules include one or both of proteins and ligands.

44. The method as in claim 43, wherein the molecular interactions include at least one of protein-ligand binding or protein-protein interaction.

45. The method as in claim 43, wherein the molecular interactions among the biomolecules alters a molecular property of at least one of the molecular-interacted biomolecules, wherein the changed molecular property includes at least one of protein folding or conformational change, protein denaturing, or protein surface charge alteration.

46. The method as in claim 41, wherein the biomolecules are not labeled and are not immobilized to a surface in the microfluidic channel.

47. The method as in claim 41, wherein the receiving the fluid sample includes sequentially receiving a first fluid sample including a first type of biomolecules and a second fluid sample including a second type of biomolecules having a slower diffusivity than the first type, wherein the processing includes determining the occurrence of molecular interactions between the first and second types of biomolecules when the change in the electrical signal includes an amplitude increase of a waveform of the first type of biomolecules.

48. A method for high-throughput detection of molecular interactions, comprising:
receiving a plurality of fluid samples including candidate molecules in an array of wells formed on a substrate;
receiving a fluid including target biomolecules in a microfluidic channel formed on the substrate in fluidic connection with the array of wells, wherein the fluid carrying the target biomolecules from the microfluidic channel to openings of the wells create fluid interfaces between the fluid and the fluid samples;
detecting a change in an electrical signal from an electrode disposed on a surface of a corresponding well, wherein the detected change in the electrical signal is based at least partly on molecular interactions between the target biomolecules and candidate molecules causing an induced surface charge on the corresponding electrode; and
processing the detected change in the electrical signal from each electrodes associated to the corresponding wells to determine an occurrence of the molecular interactions between the target biomolecules and the respective candidate molecules.

49. The method as in claim 48, wherein the receiving the fluidic samples in the array of wells and the receiving the fluid in the microfluidic channel enable the candidate molecules and the target molecules, respectively, to diffuse across the fluid interface from the corresponding wells with different diffusivities such that:
a given molecular interaction between a given target biomolecule and a given candidate molecule induces a surface charge on the corresponding electrode to change the electrical signal detected at the corresponding electrode; and
at least some of the diffusion transported candidate molecules interact with at least some of the target biomolecules proximate to or in the corresponding well.

50. The method as in claim 49, wherein the molecular interactions between the target biomolecules and the respective candidate molecules in or out of the corresponding well include binding of the candidate molecule to the target biomolecule.

51. The method as in claim 50, wherein the binding of the candidate molecules to the target biomolecules out of the corresponding well results in candidate molecules being brought back into their respective well attached to the bound target biomolecule.

52. The method as in claim 48, wherein at least some of the candidate molecules and the target biomolecules are not labeled and are not immobilized to the substrate.

53. The method as in claim 48, wherein the array of wells includes at least a hundred wells.

54. The method as in claim 48, wherein the candidate molecules and the target biomolecules include at least one of proteins or ligands.

55. The method as in claim 48, wherein the target biomolecules include proteins and the candidate molecules include drugs.
